(19) United States
(12) Patent Application Publication
(10) Pub. No.: US 2020/0402658 A1
(43) Pub. Date: Dec. 24, 2020

(54) USER-AWARE EXPLANATION SELECTION FOR MACHINE LEARNING SYSTEMS

(71) Applicant: International Business Machines Corporation, Armonk, NY (US)

(72) Inventors: Richard J. Tomsett, Southampton (GB); Graham White, Alton (GB); David S. Braines, Fareham (GB)

(21) Appl. No.: 16/447,393

(22) Filed: Jun. 20, 2019

(51) Int. Cl. G16H 50/20; G16H 50/30

(52) U.S. Cl. CPC G16H 50/20; G16H 50/30

(57) ABSTRACT

Systems, computer-implemented methods and computer program products are provided facilitating explanation selection for machine learning system output. In one embodiment, the computer-implemented method includes: accessing, by a device operatively coupled to a processor, a plurality of explanation generation components to generate different types of explanations of a machine learning output; monitoring, by the device, indicators of an entity receiving the machine learning system output to obtain user emotional state descriptors; and providing, by the device, an explanation from a selected explanation generation component to a user interface for evaluation of the machine learning system output with the explanation.

### STATEMENT REGARDING FEDERALLY SPONSORED RESEARCH OR DEVELOPMENT

This invention was made with Government support under Contract No.: W911NF-16-3-0001. These disclosures related to Defense Science and Technology Laboratory (DSTL) rather than a specific agency. The Government has certain rights in this invention.

### BACKGROUND

One or more embodiments relate to user-based explanation selection for machine learning systems, and more specifically, to explanation selection based on user emotion.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, systems, computer-implemented methods, apparatus and/or computer program products that facilitate selecting explanations from machine learning systems based on user emotion are described.

According to an aspect of the present invention there is provided a system, comprising: a memory that stores computer executable components; and a processor, operably coupled to the memory, and that executes the computer executable components stored in the memory, wherein the computer executable components comprise: an explanation selection component that accesses a plurality of explanation generation components to generate different types of explanations of a machine learning output; and a monitoring component that monitors indicators of an entity receiving the machine learning system output to obtain user emotional state descriptors, and wherein the explanation selection component also provides an explanation from a selected explanation generation component to a user interface for evaluation of the machine learning system output with the explanation.

According to another aspect there is provided a computer-implemented method that comprises: accessing, by a device operatively coupled to a processor, a plurality of explanation generation components to generate different types of explanations of a machine learning output; monitoring, by the device, indicators of an entity receiving the machine learning system output to obtain user emotional state descriptors; and providing, by the device, an explanation from a selected explanation generation component to a user interface for evaluation of the machine learning system output with the explanation.

According to another aspect there is provided a computer program product facilitating explanation selection for machine learning system output, the computer program product comprising a computer readable storage medium having program instructions embodied therewith, the program instructions executable by a processor to cause the processor to: access a plurality of explanation generation components to generate different types of explanations of a machine learning output; monitor indicators of an entity receiving the machine learning system output to obtain user emotional state descriptors; and provide an explanation from a selected explanation generation component to a user interface for evaluation of the machine learning system output with the explanation.

### BRIEF DESCRIPTION OF DRAWINGS

FIG. 1 is a schematic diagram of an example, non-limiting embodiment of a system in accordance with one or more embodiments described herein.

FIG. 2 is a flow diagram of an example, non-limiting embodiment of a computer-implemented method in accordance with one or more embodiments described herein.

FIGS. 3A and 3B are schematic diagrams of further aspects of the system in accordance with one or more embodiments described herein.

FIG. 4 is block diagram of an example, non-limiting embodiment of a system in accordance with one or more embodiments described herein.

FIG. 5 is a block diagram of an embodiment of a computer system or cloud server in which one or more embodiments of the present invention can be implemented.

FIG. 6 is a schematic diagram of a cloud computing environment in which one or more embodiments of the present invention can be implemented.

FIG. 7 is a diagram of abstraction model layers of a cloud computing environment in which one or more embodiments of the present invention can be implemented.

FIG. 8 illustrates a block diagram of an example, non-limiting machine learning system that facilitates explanation selection in accordance with one or more embodiments described herein.

FIG. 9 illustrates another block diagram of an example, non-limiting machine learning system that facilitates explanation selection in accordance with one or more embodiments described herein.

FIG. 10 illustrates a high-level block diagram of an example, non-limiting explanation generation component in accordance with one or more embodiments described herein.

FIG. 11 illustrates a high-level block diagram of an example, non-limiting explanation selection component in accordance with one or more embodiments described herein.

### DETAILED DESCRIPTION OF THE INVENTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

Entities often want an explanation for why a machine learning (ML) system produced a particular output, to help them make better decisions based on these outputs. As used herein, the term “entity” can be or represent a human, user, robot, device and/or computer. Many methods for producing different kinds of explanation have been proposed, each with benefits and drawbacks. The impact of these benefits and drawbacks on the entity decision-making will depend on the entity preferences and the mental state of the entity, which is collectively called the “user description” herein. One or more embodiments of the invention described herein can be a selection method to choose which one or more explanation methods to use depending on the user description.

One or more embodiments are directed to computer processing systems, computer-implemented methods, apparatus and/or computer program products that facilitate efficiently, effectively, and automatically (e.g., without direct human involvement) choosing explanations to present to a machine learning (ML) system user. For example, the system considers: (1) several dimensions of the entity preferences (e.g., preference for text-based explanations, preference for visual explanations) and (2) the entity current mental state (e.g., stress level, concentration level, calmness) as inferred by a user monitoring system. It selects appropriate explanations to present to the entity based on these factors.

In order to facilitate selecting an explanation for a machine learning system user, one or more embodiments described herein include a set of explanation methods that can provide different kinds of explanation for the ML system output. In one or more embodiments, there is provided a selection method for choosing the current best explanation to show to the entity. The one or more known explanation generation components can be interchangeably referred to as “explanation generators” herein. In various implementations, the known or reference explanations include metadata that has been annotated or otherwise added for usage (e.g., stored answer preferences, state of the entity) by a single entity or a group of entities. In one or more embodiments described herein, the explanation generators can explain the numeric relations that comprise the probabilistic model and explain the probabilities that are derived from the model. Hence, in order to provide an effective explanation, an explanation generator can relate its numerical information to the world knowledge underlying the numerical information.

In one or more embodiments described herein, explanation generation in the system is an interactive, multimodal process that helps entities understand the decision. Entities have different information needs. Entities interested in the model structure will be concerned with how the system justifies its answers. Entities interested in how the model relates its conclusions to their own views of the problem will be concerned with why the system answered as it did. Graphical depictions of quantitative data help people understand relative magnitudes, but graphical depictions of other relationships between abstract concepts is problematic.

In one or more embodiments described herein, entities express themselves through direct manipulation and natural language description. The system responds to the entities by presenting synchronized graphical and verbal replies that clarify and reinforce its answers. Entities concerned with the underlying structure of the model can view a graphical depiction of the model's overall topology enhanced with a natural language summary of important results. Entities can interact with the system by selecting a node for further description. Entities interested in how the results of the system relate to their concerns can interact with the systems by asking questions or making statements.

One or more embodiments described herein provides a system that engages the entity in a dialog that is sensitive to the entity needs. Explanation generation in the system is an interactive, multimodal process that helps entities understand the decision system without expertise in machine learning technology. The explanation process supports an ongoing dialog between the system participants. Entities are able to select and summarize important probabilistic relationships. The system incrementally produces multimodal explanations that use natural language, text, graphics, and animation in an ongoing dialog adapting the interaction to the entities' needs and concerns.

One or more of the computer processing systems, computer-implemented methods, apparatus and/or computer program products described herein can employ hardware and/or software to solve problems that are highly technical in nature (e.g., related to machine learning, related to automated correspondence between answer generation and explanation generated content), that are not abstract and that cannot be performed as a set of mental acts by a human. For example, a human, or even thousands of humans, cannot efficiently, accurately and effectively manually analyze the voluminous amounts of data variables gathered via computer executable components to generate explanations of a machine learning process. One or more embodiments of the subject computer processing systems, methods, apparatuses and/or computer program products can select one or more explanation generation components as a function of stored answer preferences of an entity and a detected state of the entity. By employing stored answer preferences of the entity and a detected state of the entity, the selection time and/or accuracy associated with the explanation generation process can be substantially improved. Further, one or more embodiments of the subject techniques facilitate an explanation selection component that selects one of the explanation generators. For example, by determining correspondence between the stored answer preferences and detected state of the entity, one or more embodiments of the explanation generation techniques described herein facilitate identifying the most relevant explanation to show to the entity. For instance, one or more embodiments of the subject techniques provide a selection method that considers several dimensions of the entity preferences (e.g., preference for text-based explanations or visual explanations), and the entity current mental state (e.g., stress level, concentration level, calmness) as inferred by a user monitoring system.

Further, one or more embodiments of the computer processing systems, computer-implemented methods, apparatuses and/or computer program products facilitate controlling the display of information based on entity preferences and current mental state. For example, sending text to be presented to the entity and depending on the user preferences, the text could be spoken, displayed as a word count, or any other mechanism of altering text—deciding this at the time of display in some embodiments. The source from which information should be sent to the user interface and potentially which format that information is displayed in can be gathered at the point of display and actively add more information according to user preference and current mental state.

The requirements for explanations of a machine learning output to be useful depend on the entity current mental state. The entity current mental state (e.g., calm, agitated, excited, distracted, etc.) affects how quickly and easily the entity can interpret different kinds of explanations. Where explanations require different levels of cognitive engagement, the entity mental state or emotion is important. The entity can also have preferences for explanations, which can depend on entity expertise. The user preferences can be based on factors such as the time available to process the explanations and the depth of knowledge required.

The described system is an automated system for choosing explanations to present to an entity of a machine learning system. The system considers the entity current mental state as monitored via biological and/or behavioral sensors for the entity and selects appropriate explanations to present to the entity. The entity preferences can also be taken into consideration by the automated system as well as a reaction to previous machine learning system explanations providing a feedback mechanism.

One or more embodiments relate to user-based explanation selection for machine learning systems, and more specifically, to explanation selection based on user emotion. Machine learning systems provide data analysis that automates analytical model building based on sample data known as training data in order to make predictions or decisions. Due to the automated nature of machine learning it is useful to have machine learning systems that provide a human-understandable reason for their predictions or decisions. Various methods are known that produce explanations for the output of machine learning systems in addition to the output itself. Examples of explanations range from decision trees to gradients of neural networks. The purpose of the explanations is to help entities to interpret the machine learning system output more effectively, thus improving human decision making based on these outputs.

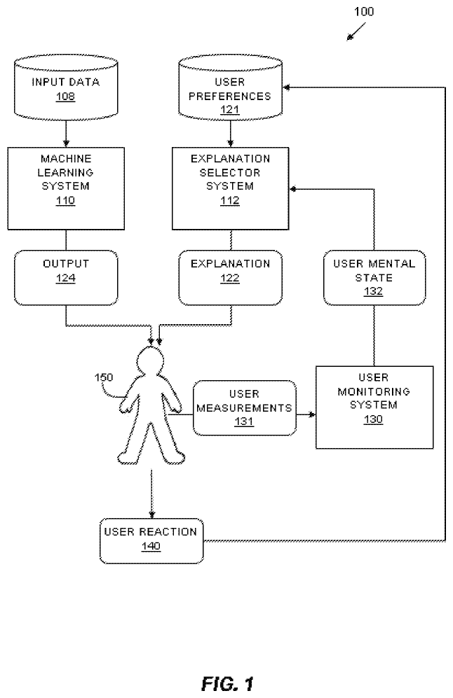

Referring to FIG. 1, an example, non-limiting embodiment of a system 100 is shown including an explanation selection component 112 for use with a machine learning system 100. A machine learning system 100 receives input data 108 and produces an output 124 for interpretation and understanding by an entity 150. The machine learning system 100 can comprise one or more pre-trained machine learning models (not shown) and a user interface (not shown). The machine learning models can be fixed or can be updated continually.

Input data 108 could be any kind of data depending on the purpose of the machine learning system (e.g. image data for an image classifier, video data for a video object detector, audio data for a speech recognition system, text data for a natural language understanding system etc).

It is noted that while FIG. 1 shows the data coming from the entity using the machine learning system, the data shown in FIG. 1 can come from any entity in various different embodiments. Further, depending on the type of model, other different types of data can be provided.

Different forms of explanation 122 can be provided of the output of the machine learning system 100. Entities often want an explanation for why a machine learning system produced a particular output to help them make better decisions based on these outputs.

The described explanation selection component 112 selects an explanation 122 based on the user description including the monitored mental state 132 of the entity 150 and, optionally, user preferences 121 of the entity 150. The user mental state 132 is monitored and evaluated by a user monitoring system 130 from user sensor measurements 131. The user preferences 121 can be configured by an entity or an administrator or can be based on a user reaction 140 to a previous output 124 and/or explanation 122 of the machine learning system 100. The explanation selection component 112 matches the user preferences 121 and user mental state 132 to types of explanation that can be provided of the machine learning output 124 as described further below.

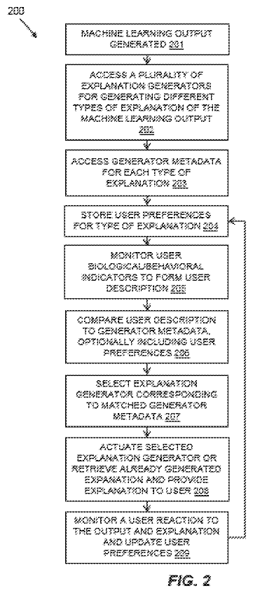

Referring to FIG. 2, a flow diagram 200 shows an example, non-limiting embodiment of the described computer-implemented method as carried out by the explanation selection component 112. Repetitive description of like elements employed in other embodiments described herein is omitted for sake of brevity.

The explanation selection component 112 can be integral to a machine learning system 100 or provided as a remote system for providing output explanation of the machine learning system 100. A machine learning system output is generated 201 in response to receiving an entity input to be modeled by the machine learning system.

The computer-implemented method can access 202 a plurality of explanation generators for generating different types of explanation of the machine learning output. The computer-implemented method can access the explanation generators before they generate an explanation for the current machine learning system output, or the computer-implemented method can access already generated explanations of the explanation generators. In various embodiments, the computer-implemented method includes accessing 203 generator metadata for each (or, one or more) explanation generators defining each type of explanation produced by that generator. The generator metadata can annotate the generator explanations in terms of user cognitive engagement, which indicates level of cognitive engagement required by the user to process the explanations. The generator metadata can be used by the explanation method selector to decide on suitable explanation methods based on the measured user mental state compared to the cognitive engagement required by the explanations.

The explanation generators can provide different types of explanations including, but not limited to, text-based explanations; visual explanations; in-depth explanations; summary explanations; analogy explanations; counterfactual explanations, etc.

In one embodiment, the multiple explanation generators can have different generator metadata for all the types of explanations generated by that explanation generator. In another embodiment, the multiple explanation generators can be executed to obtain multiple explanations of a specific machine learning system output and the explanations can be evaluated to obtain the generator metadata for the specific output.

The computer-implemented method can store and access 204 user preferences for types of explanation. The user preferences can be configured by the entity or by an administrator. User preferences can also be updated by monitoring a user reaction to a previous machine learning system output and explanation provided.

The computer-implemented method can monitor 205 user biological and/or behavioral indicators to form a user description. This can include monitoring biological indicators of: galvanic skin response; pupil dilation; heart rate; blood oxygen level; an electroencephalogram (EEG); an electromyography (EMG); behavioral patterns; etc. For example, galvanic skin response can be measured with Electrodermal Activity (EDA) sensors, pupil dilation can be measured with a camera (for example, mounted on a headset or glasses), blood oxygen level can be measured with a fingertip pulse oximeter, and behavioural patterns can be sensed using accelerometers, gyroscope, and user interactions with input devices such as a keyboard and a mouse. The entity indicators can be monitored at a current time or in advance of an entity requiring the explanations. The monitored biological indicators can be used to obtain user emotional state descriptors includes processing the biological and/or behavioral indictors to determine mental state levels of different emotions.

The computer-implemented method can compare 206 user emotional state descriptors to the cognitive engagement of the generator metadata for each explanation generator to select an explanation generator for the entity. The comparing process can include the stored user preferences. The comparing can consider several dimensions of the user preferences (for example, for types of explanation such as preference for text-based explanations, visual explanations, etc.), and the user current mental state (for example, stress level, concentration level, calmness, etc.) as inferred by a user monitoring system.

The computer-implemented method can select 207 an explanation generator corresponding to the matched generator metadata. More than one explanation generator can be selected.

The computer-implemented method can actuate 208 the one or more selected generators to provide the explanation of the machine learning system output of the generator(s). Alternatively, if the generator metadata is based on the already generated explanation, the explanation is retrieved. The one or more explanations are provided to the entity via a user interface for evaluation of the machine learning system output with the explanation.

The computer-implemented method can monitor 209 a user reaction to a machine learning system outcome explanation and provide feedback to learn and update the user preferences.

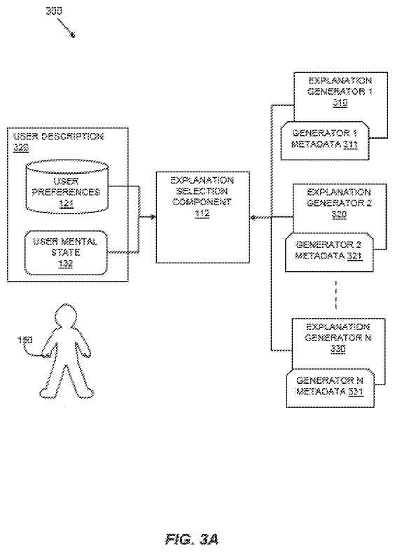

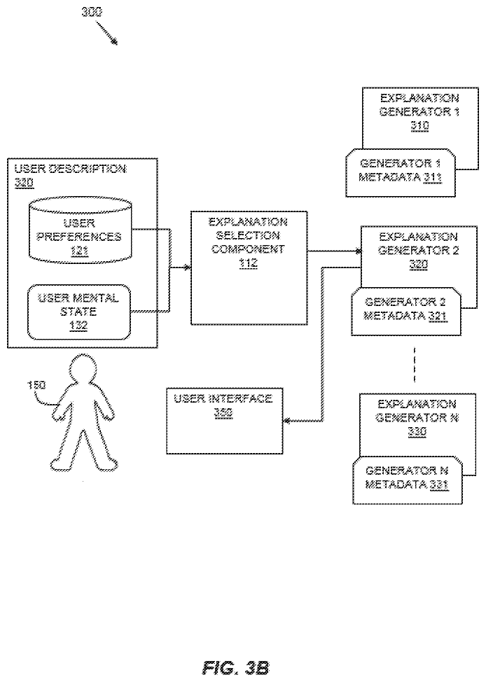

Referring to FIGS. 3A and 3B, schematic diagrams 300 illustrate an example, non-limiting embodiment of the described system and computer-implemented method. Repetitive description of like elements employed in other embodiments described herein is omitted for sake of brevity.

An explanation selection component 112 can use as input a user description 320 of an entity 150, which can be formed of a user mental state 132 as monitored from the entity 150 and user preferences 121.

Multiple explanation generators 310, 320, 330 can be available to the explanation selection component 112. The multiple explanation generators 310, 320, 330 can be generic or specific to the particular machine learning system. Each explanation generator 310, 320, 330 can be annotated with generator metadata 311, 321, 331 defining the type of explanation provided by the explanation generators 310, 320, 330. The metadata 311, 321, 331 can formally describe the generator's explanations in terms of the level of cognitive engagement required by the explanations which can be compared to a user description.

The explanation selection component 112 can match the user description 320 to the generator metadata 311, 321, 331 to select the most suitable type of explanation for the entity 150. This can be at a current time or in advance.

The descriptions in the metadata of cognitive engagement needed to comprehend the explanation can be compared against the current user description using a standard metric by, for example, combining the user mental state measures into a single cognitive engagement level and comparing against the cognitive engagement levels required by the possible explanations. Alternatively, a constraint satisfaction approach to selecting explanation generators based on the user description can be employed as described in the example below.

The explanation selection component 112 then chooses the best explanation generators according to this comparison process. The number of chosen generators can be fixed, or can be decided on by setting a threshold for the metric, or according to the user description.

As shown in FIG. 3B, an explanation generator 320 is selected by the explanation selection component 112 and the explanation provided to a user interface 350 for the entity 150. The user interface 350 can be part of the machine learning system which provides the output.

The explanation selection component 112 is paired with a machine learning system 100 and provides a selection of explanation with an appropriate cognitive engagement level for an entity based on the user's mental state and preferences.

A monitoring system can be used to take measurements of certain variables from the entity to infer their current mental state, which will affect their decision-making process, and thus the best kinds of explanation to show. The monitoring system uses these measurements to infer the user's mental state, such as their level of engagement, including distractedness, irritation, fear, anger, excitement etc. Monitoring systems of this type are known.

In addition, user preferences can also be taken into consideration in the explanation selection. User preferences include information about what kinds of explanation the entity prefers. For example, some entities can prefer text-based explanations, others image-based, others explanation by analogy etc. These preferences can be set by the entity themselves, set by an administrator who considers the entity level of expertise and familiarity with the system and task, or can be learned or refined based on the quality of decisions made by the entity given different explanations.

The selected explanation generator 320 can be actuated and run once selected. Alternatively, all the explanation generators 310, 320, 330 can be run prior to the selector choosing a specific method, thus creating various possible explanations. The explanation selector would then choose from the generated explanations. This would require more computation initially but would allow more explanations to be examined at a later date. This can be useful for forensic analysis of the system.

Cognitive state and emotion can be estimated using a variety of possible techniques using several different kinds of biosensor. For example, emotional recognition can be determined based on detection by biosensors placed on the entity. Emotional state can be inferred. The computer-implemented method can employ a wearable sensing glove incorporating photo plethysmography (PPG) sensors to record heart rate, electrodermal activity (EDA) sensors for measuring galvanic skin response, skin temperature (SKT) sensors for measuring fingertip temperature and electrocardiography (EMG) sensors for recording heart rhythm, connected to a wireless module for transmitting data. This module can also include an accelerometer and gyroscope for activity recognition, which can also be employed to estimate, for example, user agitation. The device can connect to a smartphone wirelessly using Bluetooth. The smartphone collects the input data, and transmits it for storage in the cloud using 4G, 5G or WiFi communication (or any other type of wireless or wired communications). Supervised machine learning is used to learn a model of entity physiological response to different emotions based on these sensor data. Their system is trained to detect happiness, anger, fear, disgust, and sadness, but a similar system can be trained to detect general stress, distractedness, agitation, calmness etc. more relevant to the current selection process.

An example is given below of a possible instantiation of the user description data and the explanation generator metadata for two explanation generators. In the example, the user description contains the user emotional state already inferred from the biological and behavioral measurements, so these measurements are not mentioned directly. It also contains several “user preferences” set both by the entity and the system administrator.

The explanation metadata contains information for different explanation generator types: the Local Interpretable Model-Agnostic Explanations (LIME) method, and an explanation-by-example method (which selects 1-9 examples from the training data that are the most similar examples to the user data, and returns these to show the entity). In this example instantiation, the metadata indicates the type of explanation, its characteristics, how the explanation could be displayed to the entity, and how big a cognitive effort is required by the entity to understand the explanation.

In the example instantiation, the explanation selection method uses a set of rules and a constraint satisfaction solver to select a suitable subset of explanation generators to choose. The constraints could be soft or hard; for example, an entity with restricted privileges should have a hard constraint that they should be unable to see the training data, but a user preference for a particular type of explanation method is a soft constraint, as it could be overruled given other constraints. Example rules could be:

[HARD CONSTRAINTS]
IF user_privileges IS “restricted” THEN exposes_training_data MUST BE “false”
IF user_expertise IS “low” THEN explanation_supertype MUST NOT BE “transparent”
[SOFT CONSTRAINTS]
IF stress > 0.5 THEN cognitive_engagement SHOULD BE “low”
IF concentration < 0.4 THEN explanation_preference.value SHOULD BE < 2

Given the constraints, the explanation selection method will choose the generators that have the display_options or explanation_type that match the remaining lowest ranking (according to explanation_preference.value) explanation_preference(s). The specifics of the rule system and constraint solving program can be varied.

User Description Example:

{

“user_id”: “user1”,
“user_emotional_state”: {

“stress”: “0.3”,
“agitation”: “0.6”,
“calmness”: “0.1”,
“distraction”: “0.8”,
“concentration”: “0.1”

},
“user_preferences”: {

“expertise”: {

“value”: “low”,
“learnable”: “true”,
“user_modifiable”: “true”,
“updated_by”: “admin1”

},
“privileges”: {

“value”: “restricted”,
“learnable”: “false”,
“user_modifiable”: “false”,
“updated_by”: “admin1”

},
“explanation_preference”: {

“value”: {

“0”: “text”,
“1”: “analogy”,
“2”: ''image”

},
“learnable”: “true”,
“user_modifiable”: “true”,
“updated_by”: “user1”

}

}

}

Generator Metadata Example

[

{

“generator_id”: “1”,
“generator_name”: “LIME”,
“explanation_supertype”: “post_hoc”,
“explanation_subtype”: “feature_importance”,
“explanation_characteristics”: [

“approximate”,
“linear”,
“universal”,
“black_box”,
“local”

],
“exposes_training_data”: “false”,
“display_options”: {

“heatmap”: “image”,
“ranked_list”: “text”

},
“configuration_parameters”: { },
“cognitive_engagement”: {

“heatmap”: “low”,
“ranked_list”: “medium”

}

},
{

“generator_id”: “2”,
“generator_name”: “by_example”,
“explanation_supertype”: “post_hoc”,
“explanation_subtype”: “analogy”,
“explanation_characteristics”: [

“universal”,
“black_box”,
“local”

],
“exposes_training_data”: “true”,
“display_options”: {

“original_samples”: “input_format”

},
“configuration_parameters”: {

“num_samples_to_show”: “1..9”

},
“cognitive_engagement”: {

“original_samples”: [

{

“num_samples_to_show”: “1”,
“engagement_required”: “low”

},
{

“num_samples_to_show”: “2..4”.
“engagement_required”: “medium”

},
{

“num_samples_to_show”: “5..9”,
“engagement_required”: “high”

}

]

}

}

]

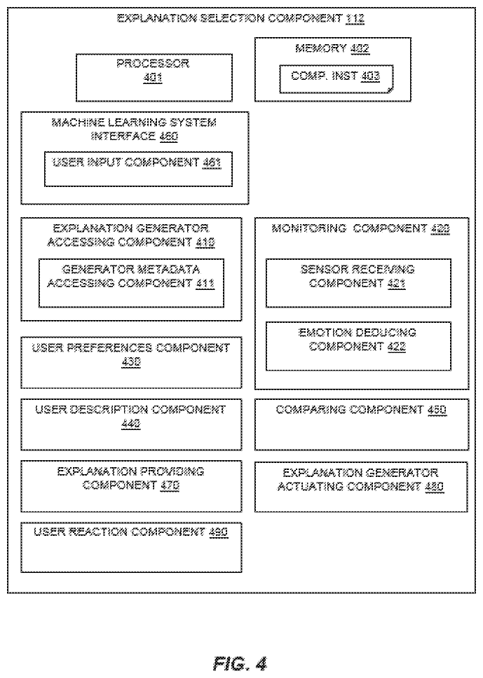

Referring to FIG. 4, a block diagram shows an example, non-limiting embodiment of the described explanation selection component 112. The explanation selection component 112 can include at least one processor 401, a hardware module, or a circuit for executing the functions of the described components which can be software units executing on the at least one processor 401. Multiple processors running parallel processing threads can be provided enabling parallel processing of some or all of the functions of the components. Memory 402 can be configured to provide computer instructions 403 to the at least one processor 401 to carry out the functionality of the components.

The explanation selection component 112 can include a machine learning system interface 460 that can cooperate with a machine learning system and a user input component 461 that can receive a user input to be modeled by the machine learning system to produce a machine learning system output for use by the entity.

The explanation selection component 112 can include an explanation generator accessing component 410 that accesses a plurality of explanation generators (as shown in FIGS. 3A and 3B) that generates different types of explanation of a machine learning output. The explanation generators can provide different types of explanations including, as examples only: text-based explanations; visual explanations; in-depth explanations; summary explanations; analogy explanations; and counterfactual explanations.

The explanation generator accessing component 410 includes a generator metadata accessing component 411 that can access generator metadata for each explanation generator, wherein the generator metadata defines each type of explanation by annotation including explanation characteristics, display options, and user cognitive engagement level required.

The explanation selection component 112 can include a monitoring component 420 that monitors biological and/or behavioral indicators of an entity receiving the machine learning system output to obtain user emotional state descriptors for a current time. The biological monitoring component 420 can include a sensor receiving component 421 that can receive biological and/or behavioral sensor readings from sensors applied to the entity and an emotion deducting component 422 that can process the biological and/or behavioral indictors to determine mental state levels of different emotions. The biological indicators from sensors can include, for example: galvanic skin response; pupil dilation; heart rate; blood oxygen level; an electroencephalogram (EEG); an electromyography (EMG) and/or behavioral patterns.

The explanation selection component 112 can also include a user preferences component 430 that can access user preferences for types of explanation that are also applied to the selection process. The explanation selection component 112 can include a user description component 440 that can provide user descriptors of the user's emotional state and based on user preferences and a comparing component 450 that can compare user emotional state descriptors to the generator metadata for each explanation generator to select an explanation generator.

The explanation selection component 112 can include an explanation providing component 470 that provide an explanation from the selected explanation generator to a user interface that can evaluate of the machine learning system output with the explanation.

The explanation selection component 112 can include an explanation generator actuating component 480 that can actuate the selected explanation generator to provide an explanation of the machine learning system output.

The explanation selection component 112 can include a user reaction component 490 that monitors user reaction to a machine learning system outcome explanation and provides feedback to learn and update user preferences.

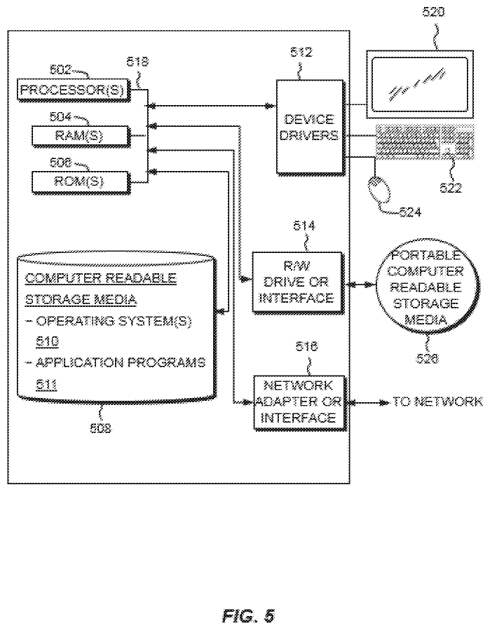

FIG. 5 depicts a block diagram of components of the computing device of explanation selection component 112, in accordance with an embodiment of the present invention. It should be appreciated that FIG. 5 provides only an illustration of one implementation and does not imply any limitations with regard to the environments in which different embodiments can be implemented. Many modifications to the depicted environment can be made.

Computing device can include one or more processors 502, one or more computer-readable RAMs 504, one or more computer-readable ROMs 506, one or more computer readable storage media 508, device drivers 512, read/write drive or interface 514, and network adapter or interface 516, all interconnected over a communications fabric 518. Communications fabric 518 can be implemented with any architecture designed for passing data and/or control information between processors (such as microprocessors, communications and network processors, etc.), system memory, peripheral devices, and any other hardware components within the system.

One or more operating systems 510, and application programs 511, are stored on one or more of the computer readable storage media 508 for execution by one or more of the processors 502 via one or more of the respective RAMs 504 (which typically include cache memory). In the illustrated embodiment, each of the computer readable storage media 508 can be a magnetic disk storage device of an internal hard drive, CD-ROM, DVD, memory stick, magnetic tape, magnetic disk, optical disk, a semiconductor storage device such as RAM, ROM, EPROM, flash memory, or any other computer readable storage media that can store a computer program and digital information, in accordance with embodiments of the invention.

Computing device can also include a R/W drive or interface 514 to read from and write to one or more portable computer readable storage media 526. Application programs 511 on computing device can be stored on one or more of the portable computer readable storage media 526, read via the respective R/W drive or interface 514 and loaded into the respective computer readable storage media 508.

Computing device can also include a network adapter or interface 516, such as a TCP/IP adapter card or wireless communication adapter. Application programs 511 on computing device can be downloaded to the computing device from an external computer or external storage device via a network (for example, the Internet, a local area network or other wide area networks or wireless networks) and network adapter or interface 516. From the network adapter or interface 516, the programs can be loaded into the computer readable storage media 508. The network can comprise copper wires, optical fibers, wireless transmission, routers, firewalls, switches, gateway computers and edge servers.

Computing device can also include a display screen 520, a keyboard or keypad 522, and a computer mouse or touchpad 524. Device drivers 512 interface to display screen 520 for imaging, to keyboard or keypad 522, to computer mouse or touchpad 524, and/or to display screen 520 for pressure sensing of alphanumeric character entry and user selections. The device drivers 512, R/W drive or interface 514, and network adapter or interface 516 can comprise hardware and software stored in computer readable storage media 508 and/or ROM 506.

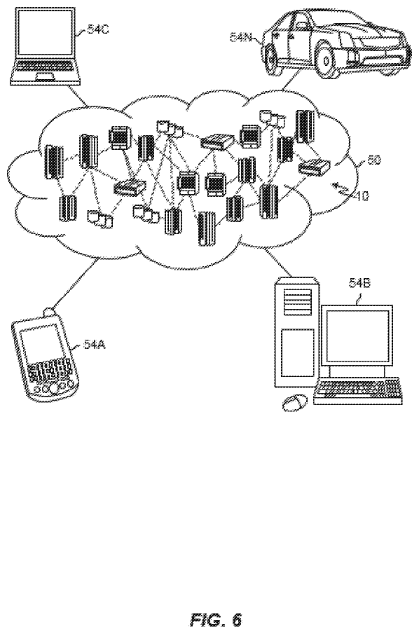

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N can communicate. Nodes 10 can communicate with one another. They can be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

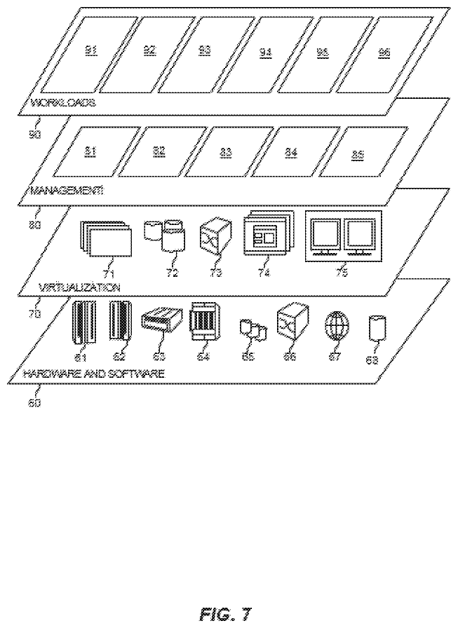

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities can be provided: virtual servers 71; virtual storage 72; virtual networks 73, including virtual private networks; virtual applications and operating systems 74; and virtual clients 75.

In one example, management layer 80 can provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources can include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment can be utilized. Examples of workloads and functions which can be provided from this layer include: mapping and navigation 91; software development and lifecycle management 92; virtual classroom education delivery 93; data analytics processing 94; transaction processing 95; and machine learning output explanation selection processing 96.

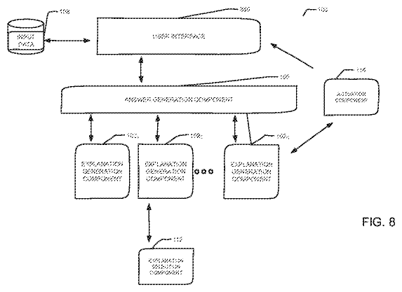

FIG. 8 illustrates a block diagram of an example, machine learning system that facilitates user-aware explanation selection for machine learning systems in accordance with one or more embodiments described herein. With reference now to FIG. 8, there is illustrated an example system 100 that can provide synchronization between explanation generation components 102_{1-N} (e.g., explanation generators) for parallel machine learning. In various embodiments, the system 100 can be a machine learning system and/or a user explanation system (e.g., the system 100 can facilitate machine learning). For example, the system 100 can perform a machine learning process (e.g., a machine learning training process) associated with a network of explanation generation components 102_{1-N} that independently perform a set of machine learning techniques to achieve one or more explanations amongst the explanation generation components 102_{1-N}. In some embodiments, the system 100 can employ learning (e.g., machine learning) to determine features, classifications and/or patterns associated with data provided to the explanation generation components 102_{1-N}. The system 100 can be or be included in speech recognition systems, image recognition systems, graphical modeling systems, bioinformatics systems, data compression systems, artificial intelligence system, authentication systems, syntactic pattern recognition systems, medical systems, health monitoring systems or the like. Similarly, any of the explanation generation components 102_{1-N} and/or the answer generation component 106 can facilitate and/or be employed in such different types of systems.

In one example, the system 100 can be a neural network (e.g., an artificial neural network, a machine learning neural network, etc.) associated with interconnected deep learning that provides an estimated functional model from a set of unknown inputs. In another example, the system 100 can be associated with a Bayesian network that provides a graphical model that represents relationships between a set of variables (e.g., a set of random variables). In yet another example, the system 100 can be associated with a hidden Markov model that models data over a continuous time interval and/or outputs a probability distribution. However, the system 100 can alternatively be associated with a different machine learning system such as, but not limited to, a clustering machine learning system, a decision tree machine learning system, an instance-based machine learning system, a regression machine learning system, a regularization machine learning system, rule learning machine learning system, etc. Furthermore, it is to be appreciated that the system 100 can be any number of different types of machine learning systems to facilitate a deep learning process associated with a network of interconnected processing components.

The system 100 and/or the components of the system 100 can be employed to use hardware and/or software to solve problems that are highly technical in nature (e.g., related to bioinformatics, authentication, compression, etc.), that are not abstract and that cannot be performed as a set of mental acts by a human. Further, some of the processes performed can be performed by specialized computers for carrying out defined tasks related to the deep learning application/subject area. The system 100 and/or components of the system can be employed to solve new problems that arise through advancements in technology, computer networks, the Internet and the like. The system 100 can provide technical improvements to deep learning systems by improving processing efficiency among processing components in a deep learning system, reducing delay in processing performed by processing components in a deep learning system, avoiding or reducing the likelihood of network bottlenecks between processing components in a deep learning system, and/or improving bandwidth utilization for a network of processing components in a deep learning system, etc.

As shown in FIG. 8, the system 100 can include explanation generation components 102_{1-N}, wherein N can be an integer. For example, the system 100 can include two or more explanation generation components 102_{1-N}, wherein a total number of explanation generators can be varied based on design criteria of a particular implementation. Each of explanation generation components 102_{1-N} (or, in some embodiments, one or more of the explanation generation components 102_{1-N}) can be or include a processing engine that processes data associated with a machine learning process (e.g., a deep learning process). For example, a explanation generation component 102_{1-N} can perform a machine learning process that is also performed on one or more other explanation generation components 102_{1-N}. In another example, an explanation generation component 102_{1-N} can perform a machine learning process that is different than another machine learning process performed on one or more other explanation generation component 102_{1-N}. A machine learning process associated with explanation generation components 102_{1-N} can include, but is not limited to, a deep Boltzmann machine algorithm, a deep belief network algorithm, a convolution neural network algorithm, a stacked auto-encoder algorithm, etc. In certain embodiments, one or more of the explanation generation components 102_{1-N} can be an artificial intelligence component. One or more of the explanation generation components 102_{1-N} explanation generation components 102_{1-N} can be similarly configured. However, in another implementation, at least one explanation generation component from the explanation generation components 102_{1-N} can be configured differently than another explanation generation component from the explanation generation components 102_{1-N}.

An explanation generation component can be associated with at least one answer generation component 106 (e.g., a central processing unit, a graphical processing unit, etc.). In various embodiments, the answer generation component 106 can be or include hardware, software (e.g., a set of threads, a set of processes, software in execution, etc.) or a combination of hardware and software that performs a computing task for machine learning (e.g., a machine learning computing task associated with received data). For example, the explanation generation components 102_{1-N} can execute deep processing threads that cannot be performed by a human (e.g., are greater than the capability of a single human mind). For example, the amount of data processed, the speed of processing of the data and/or the data types processed by explanation generation components 102_{1-N} over a certain period of time can be respectively greater, faster and different than the amount, speed and data type that can be processed by a single human mind over the same period of time. For example, data processed by explanation generation components 102_{1-N} can be raw data (e.g., raw audio data, raw video data, raw textual data, raw numerical data, etc.) and/or compressed data (e.g., compressed audio data, compressed video data, compressed textual data, compressed numerical data, etc.) captured by one or more sensors and/or one or more computing devices. Moreover, explanation generation components 102_{1-N can be fully operational towards performing one or more other functions (e.g., fully powered on, fully executed, etc.) while also processing the above-referenced deep learning data.}

In an aspect, each of the explanation generation components 102_{1-N} can be associated with, comprise and/or be operatively coupled to a computing device (e.g., a computing device associated with one or more processors). For example, first explanation generation component 1021 102₁ can be implemented on a first computing device, second explanation generator 102₂ can be implemented on the first computing device or a second computing device, third explanation generator 102₃ can be implemented on the first computing device, the second computing device or a third computing device, etc. In an implementation, more than one explanation generation component 102_{1-N} can be implemented on a computing device. For example, first explanation generation component 102₁ and second explanation generation component 102₂ can be implemented on a first computing device, third explanation generation component 102₃ can be implemented on a third computing device, etc.

One or more of the explanation generation components 102_{1-N} can be communicatively coupled to an answer generation component 106 via a user interface 350. Furthermore, each of the explanation generation components 102_{1-N} (or, in some embodiments, one or more explanation generation components 102_{1-N}) can receive at least a portion of input data 108 via the user interface 350. In an example, the explanation generation component 102₁ can receive a first portion of the input data 108 (e.g., a first set of inputs associated with the data 108), the explanation generation component 102₂ can receive a second portion of the input data 108 that is different than the first portion of the input data 108 (e.g., a second set of inputs associated with the input data 108), etc. The input data 108 can be raw, compressed and/or processed data and can include, but is not limited to, any number of different types of data such as audio data, video data, textual data and/or numerical data. In different embodiments, the sizes of the respective portions of input data 108 received by different ones of explanation generation components 102_{1-N} can be the same or different from time to time.

In an aspect, the input data 108 can be stored in a database to which the explanation generation components 102_{1-N} and/or the user interface 350 are communicatively coupled via the answer generation component 106. Therefore, in some embodiments, the explanation generation components 102_{1-N} can receive the input data 108 from a remote location via the user interface 350. The user interface 350 can include or be various wired network(s) and/or wireless network(s), including, but not limited to, local area networks (LANs), wide area networks (WANs) such as the Internet and/or networks that provide interconnections for devices associated with a defined workspace.

In some embodiments, the explanation generation components 102_{1-N} can generate a common model (e.g., a common deep learning model) based on the input data 108. The input data 108 can be, for example, training data (e.g., a training set) employed for a training process associated with performing deep learning. For example, the input data 108 can be a set of inputs for output data (e.g., a machine learning model, a neural network model, etc.) generated by the explanation generation components 102_{1-N}.

The answer generation component 106 can be associated with, comprise and/or be operatively coupled to at least one processor. In one aspect, the answer generation component 106 can be implemented on a processor that is different than a processor that includes one or more of explanation generation components 102_{1-N}. In some embodiments, the answer generation component 106 can be associated with a server device (not shown) (e.g., the answer generation component 106 can be implemented on a server device). The answer generation component 106 can be or include hardware, software (e.g., a set of threads, a set of processes, software in execution, etc.) or a combination of hardware and software.

The explanation generation components 102_{1-N} can perform a deep learning process associated with the input data 108. For example, the explanation generation components 102_{1-N} can collectively determine output data based on the input data 108. The explanation generation components 102_{1-N} can, for example, collectively train a model for a deep learning process based on the input data 108. The explanation generation components 102_{1-N} can also generate respective models (e.g., a current models) for each iteration of a deep learning process. The respective models (e.g., the current models) can represent a current state of a neural network associated with the explanation generation components 102_{1-N}. In another example, the explanation generation components 102_{1-N} can collectively determine a solution for a task associated with the input data 108. In yet another example, the explanation generation components 102_{1-N} can collectively determine features, classifications and/or patterns associated with the input data 108. In yet another example, the explanation generation components 102_{1-N} can collectively perform a set of processing acts and/or a deep learning process associated with the input data 108.

In various embodiments, one or more (or, in some embodiments, each) of the explanation generation components 102_{1-N} can generate output data based on one or more defined processes (e.g., one or more defined machine learning processes, one or more defined deep learning processes, one or more defined neural network processes, user preference, entity mental state, etc.). Output data generated by the explanation generation components 102_{1-N} can be stored locally at the explanation generation components 102_{1-N} (e.g., output data generated by the explanation generation components 102_{1-N} can be stored in at least one memory associated with or comprised within the explanation generation components 102_{1-N}). Additionally or alternatively, the output data can include a set of model parameters, a set of model weights for a model (e.g., a neural network model) associated with the input data 108 and/or a set of gradients. For example, the output data can be a set of parameters (e.g., a set of model parameters) associated with model data (e.g., a machine learning model, a neural network model, etc.). Additionally or alternatively, the output data can be a set of model weights associated with model data (e.g., a machine learning model, a neural network model, etc.). Additionally or alternatively, the output data can be a set of gradients associated with model data (e.g., a machine learning model, a neural network model, etc.).

During a first act for a deep learning process, the explanation generation components can generate respective output data based on processing the respective portions of the input data 108 received by the answer generation component 106. After performing the first act for the deep learning process, one or more of the (or, in some embodiments, each of the) explanation generation components 102_{1-N} can store respective output data in one or more respective memories operatively coupled to the explanation generation components 102_{1-N}. In an example, the explanation generation component 102₁ can generate output data based on the first portion of the input data 108 (e.g., during the first act for the deep learning process), the explanation generation component 102₂ can generate output data based on the second portion of the input data 108 (e.g., during the first act for the deep learning process), etc. As such, in some embodiments, the explanation generation components 102_{1-N} can store output data generated in response to the input data 108 rather than storage of the output data at a centralized entity (for example, at the explanation selection component 112).

The combination of the explanation generation components 102_{1-N} and the answer generation component 106 is non-obvious since the explanation selection component 112 performs novel dynamic selection of the explanation generation components 102_{1-N} during a deep learning process base on the input data (e.g., user preference and mental state.) During a deep learning process associated with the explanation generation components 102_{1-N}, the explanation selection component 112 can repeatedly select explanation generation components 102_{1-N} to groups based on processing of the explanations received by the explanation generation components 102_{1-N}. For example, the explanation selection component 112 can determine one or more explanations from the explanation generation components 102_{1-N} based on defined criterion associated with the explanation generation components 102_{1-N}, the input data 108, and/or other data associated with the answer generation component 106. Furthermore, the explanation selection component 112 can select one or more explanations from the explanation generation components 102_{1-N} multiple times during a deep learning process associated with the input data 108. In an aspect, the explanation selection component 112 can select one or more explanations from the explanation generation components 102_{1-N} based on processing of data by the explanation generation components 102_{1-N}. For example, the explanation selection component 112 can select one or more explanations from the explanation generation components 102_{1-N} based on processing time of data (e.g., a finishing time for processing data, an interval for processing the data, an amount of time for processing the data, etc.) by the explanation generation components 102_{1-N}. The explanation selection component 112 can select an explanation from the explanation generation components 102_{1-N}, in one example, based on a training process (e.g., a deep learning process, a mini-batch training process, etc.) associated with the input data 108. For example, the explanation selection component 112 can determine an explanation from the explanation generation components 102_{1-N} based on a rate (e.g., a processing speed) in which training data is processed by explanation generation components 102_{1-N}.

In a non-limiting example, the explanation selection component 112 can assign the explanation generation component 102₁ and the explanation generation component 102₃ to a particular group in response to a determination that the explanation generation component 102₁ is or will be the first explanation generation component to finish processing the input data 108, and that the explanation generation component 102₃ is or will be the next explanation generation component to finish processing the input data 108. As such, the explanation selection component 112 can employ a first-come first-served scheme to assign explanation generation components 102_{1-N} to groups. In another aspect, the explanation selection component 112 can maintain a defined list of groups for the explanation generation components 102_{1-N} before the explanation generation components 102_{1-N} receive the input data 108. As such, in this example, the explanation selection component 112 can concurrently (or in parallel) assign groups within the set of explanation generation components 102_{1-N}.

In yet another aspect, the explanation selection component 112 can form one or more explanations from the explanation generation components 102_{1-N} based on a location (e.g., a locality) of a particular explanation generation component with respect to other explanation generation components 102_{1-N} in the system 100. A location of the explanation generation component can be indicated, for example, based on an identity of a particular processor in which the processing component resides. For example, explanation generation components 102_{1-N} can be stored on multiple processors. Therefore, the explanation selection component 112 can store a list of defined processors in the system 100 and/or a subset of explanation generation components 102_{1-N} that are stored on the defined processors. In a non-limiting example, the explanation selection component 112 can assign the explanation generation component 102₁ and the explanation generation component 102₃ to the same group in response to an explanation that the explanation generation component 102₁ and the explanation generation component 102₃ are implemented on a common computing device (e.g., a common processor or a common set of processors). The one or more defined criteria (which can be the same as or different from one another) employed by the explanation selection component 112 to form groups of explanation generation components 102_{1-N} can vary during the deep learning process associated with the explanation generation components 102_{1-N}.

The explanation selection component 112 can repeatedly form groups within the explanation generation components 102_{1-N} until a defined criterion associated with the input data 108 and/or the explanation generation components 102_{1-N} is satisfied. By way of example, but not limitation, the defined criterion can be a number of groups formed by the explanation selection component 112 reaching a defined value or an error value (e.g., a training error value, etc.) associated with the input data 108 reaching a defined value. Accordingly, collaborative groups of explanations can be dynamically synchronized by the explanation selection component 112 for parallel learning during a deep learning process.

In some embodiments, the explanation selection component 112 can manage selection of explanation generation components 102_{1-N} to groups without storing and/or processing output data generated by explanation generation components 102_{1-N} (e.g., without storing and/or processing output data generated by explanation generation components 102_{1-N}). As such, the explanation selection component 112 can be configured as an arbiter rather than a parameter server device (e.g., a centralized mechanism that collects, stores and/or processes output data generated by processing components, a centralized mechanism that transmits new data to processing components to guide a direction of a task being performed by processing components, etc.).

Groups determined by the explanation selection component 112 can comprise different subsets of the explanation generation components 102_{1-N}. In various embodiments, groups determined by the explanation selection component 112 can comprise different numbers of explanation generation components 102_{1-N}. In some embodiments, the explanations in different groups can be mutually exclusive while, in other embodiments, one or more explanations assigned to a first group can also be assigned to a second group. In an aspect, during a first act for a deep learning process, the explanation selection component 112 can assign two or more explanation generation components 102_{1-N} to a group based on processing of the input data 108 by the particular explanation generation components. Output data generated by one or more (or, in some embodiments, each) of explanation generation components 102_{1-N} in the group can be exchanged with other explanation generation components 102_{1-N} in the group. Components in the machine learning system 100 can communicate with one another via the actuation component 114 and/or via one or more direct communication channels between the explanation generation components. Any number of different communication protocols can be employed.

Additionally, explanation generation components in a group can iteratively generate other output data based on data received from at least one other explanation generation component in the group. The other output data can include further processed data, for example (e.g., further processed audio, video, textual and/or numerical data; further updated model parameter, model weights and/or gradients). One or more of the explanation generation components 102_{1-N} can generate the other output data during one or more subsequent acts for the deep learning process (e.g., during one or more acts after the first act for the deep learning process).

A subsequent act for the deep learning process can be a subsequent processing act that is performed by explanation generation components 102_{1-N} after a first process act associated with the input data 108. For example, explanation generation component 102₁ can generate other output data based on the first portion of the input data 108 and/or data received from at least one other explanation generation component (e.g., during a subsequent act for the deep learning process), explanation generation component 102₂ can generate other output data based on the second portion of the input data 108 and/or data received from at least one other explanation generation component (e.g., during the subsequent act for the deep learning process), etc. The explanation generation component 102₁ can generate updated output data (e.g., new output data) based on the input data 108 (e.g., a first portion of the input data 108) and/or the other output data generated by the explanation generation component 102₂. The updated output data can be an updated version of the output data that is stored in the memory operatively coupled to the explanation generation component 102₁.

In response to selection of a second group by the explanation selection component 112, the explanation generation component 102₁ and the explanation generation component 102₃ can exchange data. For example, the explanation generation component 102₁ can transmit the updated output data to the explanation generation component 102₃. Furthermore, the explanation generation component 102₃ can transmit the data (e.g., communication data) to explanation generation component 102₁. The explanation generation component 102₁ can then perform further processing based on the other data generated by the explanation generation component 102₃. For example, the explanation generation component 102₁ can generate updated output data (e.g., new output data) based on at least a portion of the input data 108, data stored in the memory operatively coupled to the explanation generation component 102₁ and/or the data generated by the explanation generation component 102₃. The updated output data can be an updated version of the output data that is stored in the memory operatively coupled to the explanation generation component 102₁.

Then, the explanation selection component 112 can select at least the explanation generation component 102₁ and another explanation from the explanation generation components 102_{1-N} to a third group based on a defined criterion. The defined criterion associated with the third group can correspond to the defined criterion associated with the first group and/or the second group. Alternatively, the defined criterion associated with the third group can be different than (e.g., distinct from) the defined criterion associated with the first group and/or the second group. The defined criterion can be associated with the input data 108, the updated output data generated by the explanation generation component 102₁, the explanation generation component 102₁ and/or the other explanation generation component from the explanation generation components 102_{1-N}. In response to formation of the third group by the explanation selection component 112, the explanation generation component 102₁ and the other explanation generation component from the explanation generation components 102_{1-N} can exchange data, etc. The explanation selection component 112 can continue forming a group for the explanation generation component 102₁ until it is determined that a deep learning process associated with the data 108 is complete. As such, the explanation selection component 112 can repeatedly change a group to which an explanation generation component 102_{1-N} belongs during processing of the input data 108 (e.g., during a deep learning process associated with the input data 108).

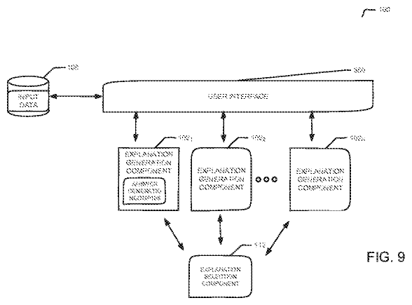

In the embodiment shown in FIG. 9, the answer generation component 106 can be implemented on at least one of the explanation generation components 102_{1-N}. For example, the answer generation component 106 can be implemented on at least the first explanation generation component 102₁. As such, the first explanation generation component 102₁ that can also include the answer generation component 106 can receive at least a portion of the input data 108 via the user interface 350, generate output data based on the input data 108, exchange data with one or more other explanation generation components from the explanation generation components 102_{2-N}, etc., as more fully described herein. Furthermore, the answer generation component 106 included in the first explanation generation component 102₁ can assign two or more explanation generation components 102_{1-N} to a group, as more fully disclosed herein. As such, since the embodiment of FIG. 9 indicates the first explanation generation component 102₁ having the functionality of the answer generation component 106, and such can be provided for any number of the explanation generation components 102_{1-N}, in some embodiments, formation of groups associated with the explanation generation components 102_{1-N} can be determined autonomously among the explanation generation components 102_{1-N} (e.g., without a separate answer generation component in communication with the explanation generation components 102_{1-N} via the user interface 350, as shown and described with reference to FIG. 8).

Combining the answer generation component 106 with at least one of the explanation generation components 102_{1-N} is non-obvious since the explanation selection component 112 performs novel dynamic selection of the explanation generation components 102_{1-N} and this embodiment allows autonomous selection of the explanation generation component 102_{1-N}. In an aspect, the explanation selection component 112 (e.g., the answer generation component 106 included in the first explanation generation component 102₁) can provide two or more explanations to a selection based on processing of the input data 108 by the explanation generation components 102_{1-N}. For example, the explanation selection component 112 can assign two or more explanations to a selection based on processing time of data (e.g., the time duration for one or more explanation generation components to complete processing of data). In another aspect, the explanation selection component 112 (e.g., the answer generation component 106 included in the first explanation generation component 102₁) can randomly select the explanation generation components to one or more explanations. In yet another aspect, the explanation selection component 112 (e.g., the answer generation component 106 included in the first explanation generation component 102₁) can select two or more explanation generation components 102_{1-N} to an explanation based on a defined list of user preferences. In yet another aspect, the explanation selection component 112 (e.g., the answer generation component 106 included in the first explanation generation component 102₁) can select two or more explanation generation components 102_{1-N} to an explanation based on the mental state of the entity. For example, two or more explanation generation component 102_{1-N} can be assigned to explanations before processing of input data 108 begins. In yet another aspect, the explanation selection component 112 (e.g., the answer generation component 106 included in the first explanation generation component 102₁) can select two or more explanation generation components 102_{1-N} to an explanation based on a location of the two or more explanation generation components with respect to other explanation generation components. For example, the two or more explanation generation components are selected to the same explanation can be implemented on a common computing device and/or implemented by a common set of processors.

One or more of the explanation generation components 102_{1-N} can receive portions of the input data 108 (e.g., the portions of the input data 108 can be the same or different in size and/or content) via the answer generation component 106 during a deep learning process associated with the input data 108. For example, the explanation generation component 102₁ can receive a first portion of the input data 108, the explanation generation component 102₂ can receive a second portion of the input data 108, the explanation generation component 102₃ can receive a third portion of the input data 108, the explanation generation component 102₄ can receive a fourth portion of the input data 108, and the explanation generation component 102₅ can receive a fifth portion of the input data 108. Furthermore, in an example, the first portion can be a first mini-batch associated with the input data 108, the second portion can be a second mini-batch associated with the input data 108, the third portion can be a third mini-batch associated with the input data 108, the fourth portion can be a fourth mini-batch associated with the input data 108, and the fifth portion can be a fifth mini-batch associated with the input data 108. One or more of (or, in some embodiments, each of) the explanation generation components 102_{1-N} can process respective portions of the input data 108 during a first act for a deep learning process associated with the input data 108.

In some embodiments, the answer generation component 106 can assign the explanation generation components 102_{1-N} to one or more groups after each processing act for the deep learning process. As such, during the deep learning process associated with the input data 108, the answer generation component 106 can repeatedly assign the explanation generation components 102_{1-N} to different groups. Assignment of the groups by the answer generation component 106 can facilitate improved execution time for the deep learning process and/or can facilitate reduced communication time among the explanation generation components 102_{1-N} during the deep learning process.

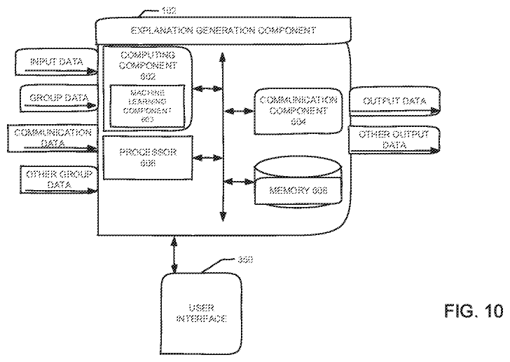

FIG. 10 illustrates a high-level block diagram of an example, non-limiting explanation generation component 102 in accordance with one or more embodiments described herein. Repetitive description of like elements employed in other embodiments described herein is omitted for sake of brevity.

The explanation generation component 102 can correspond to a processing component from explanation generation components 102_{1-N} (e.g., the explanation generation component 102 can correspond to first explanation generation components 102₁). As shown in FIG. 10, the explanation generation component 102 can also include a computing component 602 and a communication component 604. Aspects of the explanation generation component 102 can constitute machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described. In an aspect, the explanation generation component 102 can also include memory 608 that stores computer executable components and instructions. Furthermore, the explanation generation component 102 can include a processor 606 to facilitate operation of the instructions (e.g., computer executable components and instructions) by the explanation generation component 102.

The computing component 602 can receive input data (e.g., INPUT DATA shown in FIG. 10). In an aspect, the input data can correspond to the input data 108 described with reference to FIG. 8. For example, the input data can be a portion (e.g., a section) of the input data 108 and/or can be associated with a mini-batch for the input data 108. The input data can be received, for example, via the user interface 350 (also described with reference to FIG. 8). For example, the input data can be received from a database in communication with the explanation generation component 102 via the answer generation component 106. The input data can be data that is transmitted to the explanation generation component 102 and other explanation generation components associated with explanation generation components 102_{1-N}. In one example, the input data can be a portion of training data (e.g., a training set, a set of inputs, etc.) associated with a deep learning training process. Based on the input data, the computing component 602 can generate output data (e.g., OUTPUT DATA shown in FIG. 10). The output data can be generated, for example, in response to a deep learning processing (e.g., a deep learning process associated with the for the input data) that is performed by the computing component 602. In one example, the output data can be associated with a model (e.g., a current model) for a neural network associated with explanation generation components 102_{1-N}. In an aspect, the output data can be stored in the memory 608 or another memory associated with the explanation generation component 102.

In an embodiment, the computing component 602 can include a machine learning component 603. The machine learning component 603 can perform a set of machine learning computations associated with the input data 108. For example, the machine learning component 603 can perform a set of clustering machine learning computations, a set of decision tree machine learning computations, a set of instance-based machine learning computations, a set of regression machine learning computations, a set of regularization machine learning computations, a set of rule learning machine learning computations, a set of Bayesian machine learning computations and/or a set of different machine learning computations. In one example, the machine learning component 603 can perform a set of deep learning computations associated with the input data 108. For example, the machine learning component 603 can perform a set of deep Boltzmann machine computations, a set of deep belief network computations, a set of convolution neural network computations, a set of stacked auto-encoder computations and/or a set of different deep learning computations.

The explanation generation component 102 can also be in communication with the user interface 350 (described with reference to FIG. 8) via the actuation component 114. In an aspect, the explanation generation component 102 can receive group data (e.g., GROUP DATA shown in FIG. 10) from the answer generation component 106. The group data can include an indication of a group to which the explanation generation component 102 is assigned. For example, the group data can indicate that the explanation generation component 102 is assigned to a group that can also include the explanation generation component 102 and one or more other processing components associated with explanation generation components 102_{1-N}.

The communication component 604 can transmit the output data to the one or more other processing components associated with the group data. Furthermore, the communication component 604 can receive communication data (e.g., COMMUNICATION DATA shown in FIG. 10) from the one or more other processing components associated with the group data. The communication data can include a set of parameters (e.g., a set of model parameters for a model associated with the input data), a set of weights (e.g., a set of model weights for a model associated with the input data) and/or a set of gradients. Based on the communication data, the output data and/or the input data, the explanation generation component 102 can generate other output data (e.g., OTHER OUTPUT DATA shown in FIG. 10). The other output data can be an updated version of the output data. For example, the other output data can be generated based on the input data, the communication data and the output data. In one example, the explanation generation component 102 (e.g., the computing component 602) can generate an updated model by combining the model (e.g., the current model) and the communication data. The other output data can therefore represent a result of processing over the communication data, the input data and the updated model. In an aspect, the other output data can be stored in the memory 608 or another memory associated with the explanation generation component 102. In another aspect, the other output data can be combined with the output data. A combination of the output data and the other output data can therefore be stored in the memory 608 or another memory associated with the explanation generation component 102. For example, an average of the output data and the other output data can be stored in the memory 608 or another memory associated with the explanation generation component 102. In another example, a weighted version of the output data and the other output data can be stored in the memory 608 or another memory associated with the explanation generation component 102.

Moreover, the explanation generation component 102 can receive other group data (e.g., OTHER GROUP DATA shown in FIG. 10) from the answer generation component 106. The other group data can include an indication of another group for the explanation generation component 102. For example, the other group data can indicate that the explanation generation component 102 is assigned to another group that can also include the explanation generation component 102 and one or more other processing components associated with explanation generation components 102_{1-N}. The other group associated with the other group data can be different than the group associated with the group data. For example, the other group associated with the other group data can include at least one processing component that is not included in the group associated with the group data. Additionally or alternatively, the other group associated with the other group data can include a different number of processing components than the group associated with the group data. The communication component 604 can transmit the other output data to the one or more other processing components associated with the other group data. Furthermore, the communication component 604 can receive communication data (from the one or more other processing components associated with the other group data. The explanation generation component 102 can continue receiving other group data and/or communication data (e.g., other communication data) until a deep learning process associated with the input data is complete. Furthermore, the explanation generation component 102 can continue generating other output data (e.g., third output data, fourth output data, etc.) until a deep learning process associated with the input data is complete.

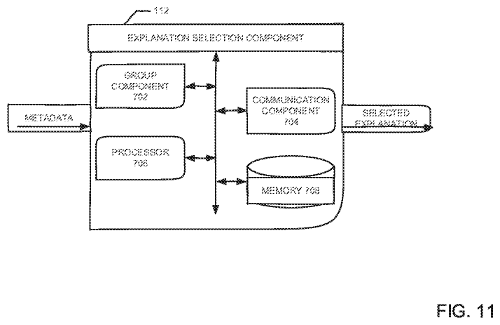

As shown in FIG. 11, the explanation selection component 112 can also include a group component 702 and a communication component 704. Aspects of the explanation selection component 112 can constitute machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described. In an aspect, the explanation selection component 112 can also include memory 708 that stores computer executable components and instructions. Furthermore, the explanation selection component 112 can include a processor 706 to facilitate operation of the instructions (e.g., computer executable components and instructions) by the explanation selection component 112.

In certain implementations, the explanation selection component 112 can receive metadata (e.g., METADATA shown in FIG. 11). The metadata can be associated with an entity preferences and/or mental state for the input data 108 and/or data processed by the explanation generation components 102_{1-N}. For example, the metadata can be indicative of a selection that a defined criterion associated with the explanation generation components 102_{1-N} and/or processing of the input data 108 by a processing component from the explanation generation components 102_{1-N} is satisfied. In an aspect, the metadata can include user preferences and/or mental state associated with processing of the input data 108 by the explanation generation components 102_{1-N}. The user preference data can be stored and processed along with the input data 108 by a processing component from the explanation generation components 102_{1-N}. Additionally or alternatively, the mental state can include (e.g., biological and/or behavioral indicators includes one or more of the group of: galvanic skin response; pupil dilation; heart rate; blood oxygen level; an electroencephalogram (EEG); an electromyography (EMG); skin temperature; behavioral patterns.) associated with explanation generation components 102_{1-N} with respect to other explanation generation component 102_{1-N}. The user preferences and mental state can indicate, for example, which explanation generation components 102_{1-N} are implemented on a common computing device and/or a common processor.

Based on the metadata, the group component 702 can generate group data (e.g., GROUP DATA shown in FIG. 10). The group data can include an indication of a group of processing components associated with explanation generation components 102_{1-N} (e.g., the group data can be indicative of an identifier for a particular group of processing components within explanation generation components 102_{1-N}). For example, the group data can indicate that two or more particular processing components is assigned to a particular group. Explanation generation components in the group determined by the group component 702 can exchange data. In an alternative implementation, the group component 702 can generate the group data without the metadata. For example, the group component 702 can randomly generate the group data (e.g., the group component 702 can randomly assign two or more processing components to a group). In another example, the group component 702 can generate the group data based on a defined group list for the explanation generation components 102_{1-N}. The communication component 704 can transmit the group data to each of the processing components associated with the group data (e.g., each of the processing components included in the group indicated by the group data). The group data transmitted by the communication component 704 can be the group data received by the explanation generation component 102 or the other group data received by the answer generation component 106.

While FIGS. 10 and 11 depict separate components in the explanation generation component 102 and explanation selection component 112, respectively, it is to be appreciated that two or more components can be implemented in a common component. Further, it is to be appreciated that the design of the explanation generation component 102 and/or the explanation selection component 112 can include other component selections, component placements, etc., to facilitate processing for parallel deep learning and/or assignment of groups for parallel deep learning. Moreover, the aforementioned systems and/or devices have been described with respect to interaction between several components. It should be appreciated that such systems and components can include those components or sub-components specified therein, some of the specified components or sub-components, and/or additional components. Sub-components could also be implemented as components communicatively coupled to other components rather than included within parent components. Further yet, one or more components and/or sub-components can be combined into a single component providing aggregate functionality. The components can also interact with one or more other components not specifically described herein for the sake of brevity, but known by those of skill in the art.

Cloud Computing

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model can include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but can be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It can be managed by the organization or a third party and can exist on-premises or off-premises.

Community cloud: the cloud infrastructure is shared by several organizations and supports a specific community that has shared concerns (e.g., mission, security requirements, policy, and compliance considerations). It can be managed by the organizations or a third party and can exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes. The present invention can be a system, a method, an apparatus and/or a computer program product at any possible technical detail level of integration. The computer program product can include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the entity computer, partly on the entity computer, as a stand-alone software package, partly on the entity computer and partly on a remote computer or entirely on the remote computer or server. In the latter scenario, the remote computer can be connected to the entity computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer executable components;
a processor, operably coupled to the memory, and that executes the computer executable components stored in the memory, wherein the computer executable components comprise:
an explanation selection component that accesses a plurality of explanation generation components to generate different types of explanations of a machine learning output; and
a monitoring component that monitors indicators of an entity receiving the machine learning system output to obtain user emotional state descriptors, and wherein the explanation selection component also provides an explanation from a selected explanation generation component to a user interface for evaluation of the machine learning system output with the explanation.

2. The system of claim 1, wherein the explanation selection component also accesses metadata for the explanation generation component, wherein the metadata defines one or more types of explanations, and wherein the metadata comprises information indicative of a level of cognitive engagement required by the entity for the one or more types of explanations.

3. The system of claim 1, wherein the indicators comprise biological or behavioral indicators, and further comprising an actuation component that actuates the selected explanation generator that provides an explanation of the machine learning system output.

4. The system of claim 3, wherein the biological and behavioral indicators comprise at least one of: a galvanic skin response; pupil dilation; a heart rate; blood oxygen level; an electroencephalogram; an electromyography; skin temperature; or a behavioral pattern.

5. The system of claim 2, wherein the metadata comprises information indicative of a level of cognitive engagement required by the entity for the type of explanation.

6. The system of claim 1, wherein the monitoring component adds content to the query, and wherein the content added is based on the entity.

7. The system of claim 1, wherein the one or more explanations are annotated with metadata that describes the explanation generation component in terms of stored answer preferences and a state of the entity.

8. The system of claim 1, wherein the computer executable components further comprise a user reaction component that monitors user reaction to a machine learning system outcome explanation and provides feedback to learn and update user preferences.

9. The system of claim 1, wherein the explanation generators provide different types of explanations comprising text-based explanations, visual explanations, in-depth explanations, summary explanations, analogy explanations, and counterfactual explanations.

10. The system of claim 1, wherein the monitoring component considers one or more preferences of the entity and a state of the entity to determine a current entity description, and detects the state of the entity based on stress level, concentration level, calmness, skin response, pupil dilation, or heart rate, and wherein the one or more preferences are set by the entity or learned by the explanation selection component based on a quality of a decision made by the entity when provided different explanations.

11. The system of claim 4, wherein the monitoring component uses a plurality of sensors to monitor galvanic skin response, pupil dilation, heart rate, blood oxygen level, electroencephalogram, electromyography, or behavioral patterns for the entity.

12. A computer implemented method, comprising;
accessing, by a device operatively coupled to a processor, a plurality of explanation generation components to generate different types of explanations of a machine learning output;
monitoring, by the device, indicators of an entity receiving the machine learning system output to obtain user emotional state descriptors; and
providing, by the device, an explanation from a selected explanation generation component to a user interface for evaluation of the machine learning system output with the explanation.

13. The computer-implemented method of claim 12, wherein the accessing comprises accessing metadata for the explanation generation component, wherein the metadata defines one or more types of explanations, and wherein the metadata comprises information indicative of a level of cognitive engagement required by the entity for the one or more types of explanations.

14. The computer-implemented method of claim 12, wherein the indicators comprise biological or behavioral indicators, and the computer-implemented method further comprising:
actuating the selected explanation generator to provide an explanation of the machine learning system output.

15. The computer implemented method of claim 12, further comprising:
comparing, by the device, employing constraint solving user emotional state descriptors to the generator metadata to match to a desired explanation type.

16. The computer implemented method of claim 15, further comprising:
accessing, by the device, user preferences for types of explanation and including the user preferences during the comparing to select an explanation generation component; and
monitoring, by the device, user reaction to a machine learning system outcome explanation and providing feedback to the entity to learn and update the user preferences.

17. The computer-implemented method of claim 14, wherein the biological and behavioral indicators comprise at least one of: a galvanic skin response; pupil dilation; a heart rate; blood oxygen level; an electroencephalogram; an electromyography; skin temperature; or a behavioral pattern.

18. A computer program product facilitating explanation selection for machine learning system output, the computer program product comprising a computer readable storage medium having program instructions embodied therewith, the program instructions executable by a processor to cause the processor to:
access a plurality of explanation generation components to generate different types of explanations of a machine learning output;
monitor indicators of an entity receiving the machine learning system output to obtain user emotional state descriptors; and
provide an explanation from a selected explanation generation component to a user interface for evaluation of the machine learning system output with the explanation.

19. The computer program product of claim 18, wherein an accessing of comprises accessing metadata for the explanation generation component, wherein the metadata defines one or more types of explanations, and wherein the metadata comprises information indicative of a level of cognitive engagement required by the entity for the one or more types of explanations.

20. The computer program product of claim 18, wherein the indicators comprise biological or behavioral indicators, and wherein the program instructions are executable by the processor to cause the processor to:
actuate the selected explanation generator to provide an explanation of the machine learning system output; and
compare employing constraint solving user emotional state descriptors to the generator metadata to match to a desired explanation type.
